(19) United States
(12) Patent Application Publication
(10) Pub. No.: US 2002/0115212 A1
(43) Pub. Date: Aug. 22, 2002

(54) Enchanced PKR expression and cytokine production

(76) Inventor: Laura A. Browning, Brentwood, CA (US)

Correspondence Address: PERKINS COIE LLP, P.O. BOX 2168, MENLO PARK, CA, 94026, US

(21) Appl. No.: 10/029,653

(22) Filed: Dec. 19, 2001

Related U.S. Application Data
(60) Provisional application No. 60/256,586, filed on Dec. 19, 2000.

(51) Int. Cl. A61K 45/00; A61K 38/21; A61K 48/00; C12N 5/08; A61K 38/19; A61K 38/20

(52) U.S. Cl. 435/366; 424/93.21; 424/85.6; 424/85.1; 424/85.2

(57) ABSTRACT

Methods for enhancing the expression of a cytokine regulatory factor and one or more cytokines in animal cell culture are described. In cell cultures that overproduce a cytokine regulatory factor, e.g., PKR, synthesis of one or more cytokines is induced to high levels, and significant amounts of individual cytokines can be recovered.

This application claims priority of U.S. Provisional Patent Application Serial No. 60/256,586 filed Dec. 19, 2000, which is incorporated in its entirety herein by reference.

### FIELD OF THE INVENTION

The present invention relates to methods and compositions for enhancing the production of cytokines in cell culture under conditions of PKR overproduction by the cells.

### REFERENCES

Altschul, S. F., et al., J Mol. Biol., 215:403-410, 1990.

Altschul, S. F., et al., Nucleic Acids Res., 25:3389-3402, 1997.

Ausubel, F. M., et al., Current Protocols in Molecular Biology, John Wiley & Sons, New York, N.Y. 1993.

Balkwill F. R. and Burke F., Immunology Today, 10(9):299, 1989.

Buffet-Janvresse, et al., J Interferon Res., 6:85-96,1986.

Cai and Williams, J. Biol. Chem., 273: 11274-11280, 1998.

Chong, K. L., et al., EMBO J., 11(4):1553-62, 1992.

Chomczynski P. et al., Analyt. Biochem. 162:156-159, 1987.

Clark, S. and Kamen, R., Science, 236:1229-1237, 1987.

Clemens, M. J. and Bommer, U. A., Int. J. Biochem. Cell Biol, 31(1):1-23, 1999.

Clemens, M. J. and Elia, A., J. Interferon Cytokine Res., 17(9):503-24, 1997.

Der, D., and Lau, A. S., Proc. Natl. Acad. Sci., USA, 92:8841-8845, 1995.

Donze, O., et al., Virol, 256:322-329, (1999).

Farrel, et al, Cell, 11:187-200, 1977.

Feng, G. S., et al, Proc. Natl. Acad. Sci., USA 89(12):5447-51 (1992).

Galabru, J., and Hovanessian, A., J. Biol. Chem., 262:15538-15544, 1987.

Gunnery, S. and Mathews, M. B., Methods, A Companion to: METHODS IN ENZYMOLOGY, 15(3):189-98, 1998.

Hershey, J. W. B., Ann. Rev. Biochem., 60: 717-755, 1991.

Hovanessian, Biochimie. 62:775-778, 1980.

Jagus, R., et al, Int. J. Biochem. Cell Biol., 31(1):123-38, 1999.

Koromilas, et al., Science, 257:1685, 1992.

Krust, et al., Virology, 120:240-246, 1982.

Kumar, A., et al., Proc. Natl. Acad. Sci., USA 91:6288-6292, 1994.

Kuhen, K. L., et al., Genomics, 36: 197-201, 1996.

Kuhen, et al, Gene, 178:191-193, 1996.

Larrick, J. W., and Wright, S. C., FASEB J., 4:3215-3223, 1990.

Levin, et al., Proc. Natl. Acad. Sci. USA, 75:1121-1125, 1978.

Liddil, J. D., et al., Cancer Res. 49:2722-2728, 1989.

Meurs, E., et al., Molecular Cell, 62: 379-390, 1990.

Mulligan and Berg, Science, 209:1422, 1980.

Murray, E., et al., Nuc. Acids Res., 17:477-508, 1989.

Orrenius, S., J. Intern. Med., 237:529-536, 1995.

Rubin, B. Y., et al., Cancer Res., 48:6006-6010, 1988.

Sambrook, et al., MOLECULAR CLONING: A LABORATORY MANUAL (Third Edition), Cold Spring Harbor Press, Plainview, N.Y., 2001.

Southern and Berg, J. Molec. Appl. Genet., 1:327, 1982.

Stellar, H., Science, 267:1445-1449, 1995.

Sugden, et al, Mol. Cell. Biol., 5:410413, 1985.

Tan, S. L., Gale, M. J., Jr., Katze, M. G., Mol. Cell. Biol, 18(5):2431-43, 1998.

Vaux, D. L., Proc. Natl. Acad. Sci. USA, 90:786-789, 1993.

Wek, R. C., Trends Biochem. Sci., 19: 491-496, 1994.

Williams, B. R., Oncogene, 18(45):6112-20, 1999.

Wong, G. and Clark, S., Immunology Today, 9(5):137, 1988.

Wu, S. and Kaufman, R. J., J Biol. Chem., 272(2):1291-6, 1997.

Yeung, M. C., et al., Proc. Natl. Acad. Sci. USA, 93,12451-12455, 1996.

Yeung, M. C., et al., Proc. Natl. Acad. Sci. USA, 96(21):11860-5, 1999.

Zamanian-Daryoush, M., et al, Oncogene, 18: 315-326, 1999.

Zamanian-Daryoush, et al, Molecular and Cellular Biology, 20:1278-1290, 2000.

### BACKGROUND OF THE INVENTION

dsRNA-activated inhibitor is a serine/threonine kinase of molecular mass 68 and 65 kDa in human and mouse cells, respectively. Binding to dsRNA or to single-stranded RNA leads to activation and autophosphorylation of PKR (Galabru and Hovanessian, 1987; Meurs, et al., 1990). This allows PKR to phosphorylate its natural substrate, the alpha subunit of eukaryotic protein synthesis initiation factor 2, eIF-2, leading to the inhibition of protein synthesis.

The nucleotide sequence of the human PKR gene has been determined and shows that the gene contains 17 exons, which encode the 551-amino acid PKR protein (Kuhen et al., 1996).

Analogous enzymes have been described in rabbit reticulocytes, different murine tissues, and human peripheral blood mononuclear cells (Farrel, et al., 1997; Levin, et al., 1978; Hovanessian, 1980; Krust, et al., 1982; Buffet-Janvresse, et al., 1986).

PKR has been shown to play a variety of important roles in the regulation of translation, transcription, and signal transduction pathways through its ability to phosphorylate protein synthesis initiation factor eIF2, and I-kappaB (the inhibitor of NF-kappaB; Kumar A, et al., 1994), in addition to other substrates.

The best characterized in vivo substrate for PKR is the alpha subunit of eukaryotic initiation factor-2 (eIF-2alpha), which, once phosphorylated, ultimately leads to inhibition of cellular and viral protein synthesis (Hershey, 1991). PKR has been demonstrated to phosphorylate initiation factor eIF-2 alpha in vitro when activated by double-stranded RNA (Chong, et al., 1992).

Activities attributed to PKR include a role in (1) mediating the antiviral and anti-proliferative activities of IFN-alpha and IFN-beta, (2) the response of uninfected cells to physiologic stress, and (3) cell growth regulation (Clemens, M. J. and Elia, A., 1997; Zamanian-Daryoush, M., et al., 1999).

It has also been suggested that PKR may function as a tumor suppressor and inducer of apoptosis. (See, e.g., Clemens M. J. and Bommer U. A., 1999; Yeung, et al., 1996; Koromilas, et al., 1992), with recent results indicating that expression of an active form of PKR triggers apoptosis, possibly through upregulation of the Fas receptor (Donze, O., et al., 1999).

It has further been shown that when a cell line capable of producing interferon is transfected with an expression vector encoding PKR, overexpression of PKR induces increased production of interferon (WO 97/08324). The interferon proteins are a family of prototypes of cytokines and have been shown to play key roles in immune defense against pathogens and cancerous tissues.

Cytokines are regulatory molecules that exhibit a range of biological activities and act on a wide range target cells. (See, e.g., Balkwill, F. R. and Burke, F., 1989; Wong, G. and Clark, S., 1988; and Clark, S. and Kamen, R., 1987.)

In general, cytokines are currently produced by the expression of recombinant proteins in insect, bacterial or mammalian host cells.

In general, cytokines and other proteins are produced for various therapeutic applications by either purifying the natural protein from cell culture or recombinantly producing the protein in insect, microbial or human cells. Natural cytokines and other proteins, produced by human cells, are preferable in that they are known to contain the full repertoire of native forms of a given cytokine or protein and have the proper protein structure including glycosylation and post translational modifications that may be unique to the protein produced in human cells. However, they are expensive and time-consuming to produce human cells.

Recombinantly produced cytokines and other proteins are less expensive to make, but dependent upon the source may contain foreign antigens, resulting in an immune response by the human subject to which they are administered, or may be less active due to structural variation from the native form, i.e., glycosylation pattern.

Present methods utilize expression of cytokines in microbial systems, which do not permit the glycosylation and native folding of the cytokine proteins, or in human cells that generally produce very low levels of recombinant protein. Thus, a method for enhancing the production of one or more cytokines in cells that produce such one or more cytokines in nature would be advantageous.

The present invention is directed to the discovery that cytokine production is enhanced in cultured mammalian cells that overexpress a cytokine regulatory factor such as PKR, IRF-3 or IRF-7.

### SUMMARY OF THE INVENTION

The present invention provides a method for enhanced cytokine production by mammalian cells that over express a cytokine regulatory factor such as PKR, IRF-3 or IRF-7.

The invention further provides cytokine regulatory factor over expressing cell line compositions that exhibit enhanced production of one or more cytokines.

In one preferred approach, a parental cell line capable of expressing PKR and one or more cytokines is provided, and subjected to limiting dilution cloning in order to generate a plurality of subclones. Subclones that exhibit at least a 2-fold (2×) greater PKR expression level than the parental cell line are selected and grown to produce a PKR over expressing cell line.

In another preferred approach, a parental cell line capable of expressing PKR and one or more cytokines is provided together with a heterologous nucleic acid construct comprising the coding sequence for a biologically active form of human PKR and the coding sequence for a selectable marker. In practicing the method, the heterologous nucleic acid construct is introduced into the parental cell line resulting in expression of PKR and the selectable marker. The cell line is then cultured in culture medium containing a selection agent and cells are selected that exhibit a PKR expression level which is at least 2-fold (2×) greater than the PKR expression level of the non-transformed parental cell line.

In either approach, the method further includes priming the PKR over expressing cells, or priming and treating the PKR over expressing cells, and growing the cells in a manner effective to generate a PKR over expressing cell line characterized by enhanced expression of one or more cytokines.

“Priming” may be accomplished by exposing the cells to a priming agent such as phorbal myristate acetate (PMA) or sodium butyrate. Examples of “treating” include non-viral induction with ds RNA and viral induction with Sendai virus or Herpes simplex.

Any of a number of cytokines may be produced by and collected from such PKR over expressing cells using the methods of the invention and include, but are not limited to, α-(interferon, β-interferon, TNF-β, IL-6 and IL-8, IL-10, IFN-gamma, IL-12 and GM-CSF.

These and other objects and features of the invention will become more fully apparent when the following detailed description is read in conjunction with the accompanying figures and examples.

### BRIEF DESCRIPTION OF THE FIGURES

FIG. 1A shows a Northern blot analysis of PKR expression following poly I:C induction in Namalwa PKR++41.027 cells and in wild type Namalwa cells (WT). (Top panel, Lanes 1 and 3—RNA isolated from resting cells, Lane 2 and 4—RNA from poly I:C treated cells; and the bottom panel presents the gel stained with ethidium bromide).

FIG. 1B shows a Northern blot analysis of PKR expression following Sendai virus (SV) induction in Namalwa PKR++41027 cells and in wild type Namalwa cells (WT). (Top panel, Lane 1—RNA from SV infected Nam-WT cells; Lane 2—RNA from SV infected Namalwa PKR++41.027 cells; and the bottom panel presents the gel stained with ethidium bromide).

FIG. 2A shows the autoradiographic image of an SD S-PAGE PKR autokinase activity assay conducted with Namalwa-WT and Namalwa-PKR++41.027 cell lines after exposing PMA for 20 hrs alone or to PMA followed by Poly I:C/DEAE Dextran for 48 hr. (Lanes 1-3—present samples which were primed with PMA but were not induced with Poly I:C/DEAE Dextran; Lanes 4-6 present samples which were primed with PMA followed by induction with Poly I:C and DEAE Dextran for 48 hr.

FIG. 2B shows the PKR kinase activities in wild type Namalwa cells (WT), and in Namalwa-PKR++41.027 subcloned progeny cell lines 41.027.A9 and 41.027.C1 after 20 hr exposure to PMA alone or to PMA followed by Poly I:C/DEAE Dextran for 48 hr.

FIG. 2C shows the fold increase of PKR kinase activity in wild type Namalwa cells (WT), and in Namalwa-PKR++41.027 subcloned progeny cell lines 41.027.A9 and 41.027.C1 after 20 hr exposure to PMA alone or to PMA followed by Poly I:C/DEAE Dextran for 48 hr.

FIG. 3A shows the fluorescent Western blot analysis of PKR expression in wild type (WT) and Namalwa-PKR++41.027 subcloned progeny cell lines 41.027.A9 and 41.027.C1 after 20 hr exposure to PMA alone or to PMA followed by Poly I:C/DEAE Dextran for 48 hr. (Lanes 1-3—present samples which were primed with PMA but were not induced with Poly I:C/DEAE Dextran; Lanes 4-6 present samples which were primed with PMA and followed by induction with Poly I:C and DEAE Dextran for 48 hr.

FIG. 3B shows the relative levels of PKR protein in wild type Namalwa cells (WT), and of Namalwa-PKR++41.027 subcloned progeny cell lines 41.027.A9 and 41.027.C1 after 20 hr exposure to PMA alone or to PMA followed by Poly I:C/DEAE Dextran for 48 hr..

FIG. 3C shows the fold increase of PKR protein in wild type Namalwa cells (WT), and in Namalwa-PKR++41027 subcloned progeny cell lines 41.027.A9 and 41.027.C1 after 20 hr exposure to PMA alone or to PMA followed by Poly I:C/DEAE Dextran for 48 hr.

FIG. 4A shows the specific activity of PKR protein in wild type Namalwa cells (WT), and in Namalwa-PKR++41.027 subcloned progeny cell lines 41.027.A9 and 41.027.C1 after 20 hr exposure to PMA alone or to PMA followed by Poly I:C/DEAE Dextran for 48 hr.

FIG. 4B shows the fold increase of the PKR specific activity in wild type Namalwa cells (WT), and Namalwa-PKR++41.027 subcloned progeny cell lines 41.027.A9 and 41.027.C1 after 20 hr exposure to PMA alone or to PMA followed by Poly I:C/DEAE Dextran for 48 hr.

FIG. 5 illustrates the IFN-alpha production in wild type Namalwa cells (WT) and Namalwa PKR++41.027 cells following PMA priming and Sendai virus induction for 24 or 48 hr, (data set 1-3) and in wild type Namalwa cells (WT) and in Namalwa PKR++41.027 cells following PMA priming and Sendai virus induction for 24 hrs in the presence of 5 mM of 2-aminopurine (2-AP) (data set 4). The figure further illustrates IFN-alpha production in wild type Namalwa cells (WT) and in Namalwa PKR++41.027 cells following PMA priming and polyI:C induction for 72 hours (data set 5) and in wild type Namalwa cells (WT) and Namalwa PKR++41.027 cells following PMA priming and polyI:C induction for 72 hours in the presence of 5 nM of 2-aminopurine (data set 6).

FIG. 6 illustrates a summary of the results of an analysis of TNF-beta, IL-6 and IL-8 cytokine levels in cultures of a Namalwa wild type (WT) and a Namalwa PKR over expressing cell line (41.027 ).

### DETAILED DESCRIPTION OF THE INVENTION

I. Definitions

Unless otherwise indicated, all technical and scientific terms used herein have the same meaning as they would to one skilled in the art of the present invention. Practitioners are particularly directed to Sambrook et al., 1989, and Ausubel, F. M. et al., 1993, for definitions and terms of the art. It is to be understood that this invention is not limited to the particular methodology, protocols, and reagents described, as these may vary.

All publications cited herein are expressly incorporated herein by reference for the purpose of describing and disclosing compositions and methodologies that might be used in connection with the invention.

A “heterologous” nucleic acid construct or sequence has a portion of the sequence that is not native to the cell in which it is expressed. Heterologous, with respect to a control sequence refers to a control sequence (i.e. promoter or enhancer) that does not function in nature to regulate the same gene the expression of which it is currently regulating. Generally, heterologous nucleic acid sequences are not endogenous to the cell or part of the genome in which they are present, and have been added to the cell, by infection, transfection, microinjection, electroporation, or the like. A “heterologous” nucleic acid construct may contain a control sequence/DNA coding sequence combination that is the same as, or different from a control sequence/DNA coding sequence combination found in the native cell.

The terms “vector”, as used herein, refer to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. A cloning or expression vector may comprise additional elements, for example, the expression vector may have two replication systems, thus allowing it to be maintained in two organisms, for example in human cells for expression and in a prokaryotic host for cloning and amplification. Cloning and expression vectors will typically contain a selectable marker.

As used herein, the term “selectable marker-encoding nucleotide sequence” refers to a nucleotide sequence that is capable of expression in mammalian cells and where expression of the selectable marker confers to cells containing the expressed gene the ability to grow in the presence of a corresponding selection agent.

As used herein, the term “promoter” refers to a nucleic acid sequence that functions to direct transcription of a downstream gene. The promoter will generally be appropriate to the host cell in which the target gene is being expressed. The promoter together with other transcriptional and translational regulatory nucleic acid sequences (also termed “control sequences”) are necessary to express a given gene. In general, the transcriptional and translational regulatory sequences include, but are not limited to, promoter sequences, ribosomal binding sites, transcriptional start and stop sequences, translational start and stop sequences, and enhancer or activator sequences. A promoter may be constitutive or inducible and may be a naturally occurring, engineered or hybrid promoter.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

As used herein, the term “operably linked” relative to a recombinant DNA construct or vector means nucleotide components of the recombinant DNA construct or vector that are directly linked to one another for operative control of a selected coding sequence. Generally, “operably linked” DNA sequences are contiguous, and, in the case of a secretory leader, contiguous and in reading frame. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “gene” means the segment of DNA involved in producing a polypeptide chain, which may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “sequence identity” means nucleic acid or amino acid sequence identity in two or more aligned sequences, aligned using a sequence alignment program.

The term “% homology” is used interchangeably herein with the term “% identity” herein and refers to the level of nucleic acid or amino acid sequence identity between two or more aligned sequences, when aligned using a sequence alignment program. For example, as used herein, 80% homology means the same thing as 80% sequence identity determined by a defined algorithm, and accordingly a homologue of a given sequence has greater than 80% sequence identity over a length of the given sequence. Exemplary levels of sequence identity include, but are not limited to, 80, 85, 90 or 95% or more sequence identity to a PKR sequence, as described herein.

Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at http://www.ncbi.nlm.nih.gov/BLAST/”. See, also, Altschul, S. F., et al., 1990 and Altschul, S. F., et al., 1997.

Sequence searches are typically carried out using the BLASTN program when evaluating a given nucleic acid sequence relative to nucleic acid sequences in the GenBank DNA Sequences and other public databases. The BLASTX program is preferred for searching nucleic acid sequences that have been translated in all reading frames against amino acid sequences in the GenBank Protein Sequences and other public databases. Both BLASTN and BLASTX are run using default parameters of an open gap penalty of 11.0, and an extended gap penalty of 1.0, and utilize the BLOSUM-62 matrix. [See, Altschul, et al., 1997.]

A preferred alignment of selected sequences in order to determine “% identity” between two or more sequences, is performed using for example, the CLUSTAL-W program in MacVector version 6.5, operated with default parameters, including an open gap penalty of 10.0, an extended gap penalty of 0.1, and a BLOSUM 30 similarity matrix.

A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm−5° C. (5° below the Tm of the probe); “high stringency” at about 5-10° below the Tm; “intermediate stringency” at about 10-20° below the Tm of the probe; and “low stringency” at about 20-25° below the Tm. Functionally, maximum stringency conditions may be used to identify sequences having strict identity or near-strict identity with the hybridization probe; while high stringency conditions are used to identify sequences having about 80% or more sequence identity with the probe.

Moderate and high stringency hybridization conditions are well known in the art (see, for example, Sambrook, et al, 1989, Chapters 9 and 11, and in Ausubel, F. M., et al., 1993, expressly incorporated by reference herein). An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

As used herein, “recombinant” includes reference to a cell or vector, that has been modified by the introduction of a heterologous nucleic acid sequence or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention.

As used herein, the terms “transformed”, “stably transformed” or “transgenic” with reference to a mammalian cell means the mammalian cell has a non-native (heterologous) nucleic acid sequence integrated into its genome which is maintained through two or more generations.

As used herein, the term “expression” refers to the process by which a polypeptide is produced based on the nucleic acid sequence of a gene. The process includes both transcription and translation.

It follows that the term “PKR expression” refers to transcription and translation of the PKR gene, the products of which include precursor RNA, mRNA, polypeptide, post-translation processed polypeptide, and derivatives thereof, including PKRs from other species such as murine or simian enzymes. By way of example, assays for PKR expression include autophosphorylation assays, assay for eIF2α phosphorylation, Western blot for PKR protein, as well as Northern blot analysis and reverse transcriptase polymerase chain reaction (RT-PCR) assays for PKR mRNA.

“Alternative splicing” is a process whereby multiple polypeptide isoforms are generated from a single gene, and involves the splicing together of non-consecutive exons during the processing of some, but not all, transcripts of the gene. Thus a particular exon may be connected to any one of several alternative exons to form messenger RNAs. The alternatively-spliced mRNAs produce polypeptides (“splice variants”) in which some parts are common while other parts are different.

As used herein, the terms “biological activity of PKR” and “biologically active PKR” refer to any biological activity associated with PKR, or any fragment, derivative, or analog of PKR, such as enzymatic activity, in particular, autophosphorylation activity and eukaryotic translation initiation factor 2 (eIF-2) phosphorylation activity.

As used herein, the terms “normal level of PKR activity” and “normal level of PKR expression” refer to the level of PKR activity or expression, determined to be present in unselected, unstimulated or uninfected cells of a particular type, for example, a particular cell line. It will be appreciated that such “normal” PKR activity or expression, is reported as a range of PKR activity or expression which is generally observed for a given type of cells (or cell line) that have not been selected for, do not include an introduced PKR coding sequence (of heterologous or autologous origin), are unstimulated (not induced or primed) and are uninfected.

The range of “normal” PKR activity or expression may vary somewhat dependent upon culture conditions. For example, the U937 cell line may have a normal range of PKR activity, which differs from the normal range of PKR activity for the Vero or Namalwa cell lines. It follows that overexpression of PKR means an expression level which is above the normal range of PKR expression generally observed for a given type of cells that have not been selected for, do not include an introduced PKR coding sequence (of heterologous or autologous origin), are unstimulated (not induced or primed) and are uninfected.

The terms “PKR overexpression” and “enhanced PKR expression” as used herein refer to a level of PKR activity, expression or production that is greater than the level of PKR expression or production typically present in cells of a particular type which have not been selected or treated in a manner effective result in PKR overexpression, as further detailed below. Accordingly, “overexpression” of PKR means a range of PKR activity, expression or production which is greater than that generally observed for a given type of cells that have not been selected for, do not include an introduced PKR coding sequence (of heterologous or autologous origin), are unstimulated (not induced or primed) and are uninfected.

As used herein, the terms “biological activity” and “biologically active”, refer to the activity attributed to a particular protein in a cell line in culture. It will be appreciated that the “biological activity” of such a protein may vary somewhat dependent upon culture conditions and is generally reported as a range of activity. Accordingly, a “biologically inactive” form of a protein refers to a form of the protein, which has been modified in a manner, which interferes with the activity of the protein as it is found in nature.

In one preferred aspect, PKR overexpression means a level of PKR activity, expression or production that is at least 150% (1.5-fold or 1.5×), preferably at least 200%, 300% or 400%, or 500% or more greater than the normal level of PKR activity, expression or production for the same cell line under the particular culture conditions employed. In other words, a cell line that over expresses PKR typically exhibits a level of PKR production or expression that is at least 1.5-fold and preferably 2-fold (2×), 3-fold (3×), 4-fold (4×), 5-fold (5×) or more greater than the level of PKR expression or production typically exhibited by the same type of cells which have not been selected or treated in a manner effective result in PKR overexpression. In some cases, a cell line that over expresses PKR exhibits a level of PKR expression or production that is 10-fold (10×) or more greater than the level of PKR expression or production typically exhibited by the same type of cells under the particular culture conditions employed and which have not been selected or treated in a manner effective result in PKR overexpression. In general, the term “treated in a manner effective result in PKR overexpression”, means introduction of a PKR coding sequence (of heterologous or autologous origin) into the cell, stimulation (priming or priming and induction) and/or infection.

As used herein, the terms “normal level of cytokine” and “normal level of protein”, relative to activity, expression, and production, refer to the level of cytokine or other protein activity, expression or production, determined to be present in cells of a particular type which have not been treated in a manner effective result in PKR overexpression. Examples include, a wild type cell line, which has not been selected or treated in a manner to result in enhanced PKR activity, expression or production and a cell line, which does not comprise an introduced PKR coding sequence. It will be appreciated that such “normal” cytokine or other protein activity, expression, or production, is reported as a range of activity, expression, or production, typically observed for a given type of cells and may vary somewhat dependent upon culture conditions.

As used herein the terms “purified” and “isolated” generally refer to molecules, either polynucleotides or polypeptides that are separated from other components of the environment in which they were found or produced. For example an isolated or purified polynucleotide or polypeptide has typically been separated from 75% or more of the components of the environment in which they were found or produced. An isolated or purified polynucleotide or polypeptide has preferably been separated from at least 80% to 85% and more preferably at least 90% or more of the components of the environment in which they were found. For example, a “purified” or “isolated” cytokine means the cytokine has been separated from at least 75% or more, preferably from at least 80% to 85% or more and more preferably from at least 90% or more of the components in the cell culture medium in which they were produced.

The terms “apoptotic cell death”, “programmed cell death” and “apoptosis”, as used herein refer to any cell death that results from, or is related to, the complex cascade of cellular events that occur at specific stages of cellular differentiation and in response to specific stimuli. Apoptotic cell death is characterized by condensation of the cytoplasm and chromatin condensation in the nucleus of dying cells. The process is associated with fragmentation of DNA into multiples of 200 base pairs and degradation of RNA as well as proteolysis in an organized manner without sudden lysis of the cell as in necrotic cell death.

As used herein, the term “inhibit apoptotic cell death”, means to partially or completely inhibit the cell death process over the time period a cell line is cultured for the purpose of cytokine or other protein expression or production. Such inhibition generally means the amount of apoptotic cell death is decreased by at least 20%, preferably by at least 50% and more preferably by 80% or more relative to the amount of apoptotic cell death observed in a cell line which has not been modified in a manner effective to inhibit apoptosis.

In the case of PKR and/or cytokine production, such inhibition generally means the amount of apoptotic cell death is decreased by at least 20%, preferably by at least 50% and more preferably by 80% or more relative to the amount of apoptotic cell death observed in a PKR-overexpressing cell line which has not been modified in a manner effective to inhibit apoptosis.

II. PKR

PKR is the only identified dsRNA-binding protein known to possess a kinase activity. PKR is a serine/threonine kinase whose enzymatic activation requires dsRNA binding and consequent autophosphorylation (Meurs, et al., 1990; Feng, G. S., et al, 1992).

Various functions have been attributed to PKR, including, phosphorylation of eukaryotic initiation factor-2 (eIF-2alpha), which, once phosphorylated, leads to inhibition of protein synthesis (Hershey, et al., 1991). This particular function of PKR has been suggested as one of the mechanisms responsible for mediating the antiviral and anti-proliferative activities of IFN-alpha and IFN-beta. An additional biological function for PKR is its putative role as a signal transducer, for example, by can phosphorylation of IkB, resulting in the release and activation of nuclear factor kB (NF-kB) (Kumar A., et al., 1994).

It has previously been demonstrated that PKR mediates the transcriptional activation of IFN expression (Der, D. and Lau, A. S., 1995). Consistent with this observation, suppression of endogenous PKR activity by transfecting U937 cells with antisense to PKR or expression of a PKR-deficient mutant resulted in diminished induction of IFN in response to viral infection (Der, D. and Lau, A. S., 1995).

In summary, PKR has been associated with (1) signal transduction for complex receptor systems (including IFN, TNF and Fas), (2) transcriptional activation of cytokine genes, (3) initiation of apoptosis, and (4) inhibition of protein synthesis by phosphorylating eIF-2α.

PKR is used herein as an example of a protein capable of regulating cytokine expression, however it will be understood that other cytokine regulatory factors may be used in place of PKR, e.g., PMA, protein kinase C (PKC) inducers, interferon-γ, interferon-α, interferon-β, IFN regulatory factors (IRFs), TNF-α, GM-CSF, EGF and PDGF.

By increasing the expression/activity of a cytokine regulatory factor, such as PKR in mammalian cells, cytokine production can be increased. Mammalian cell cultures which express a higher constitutive level of the cytokine regulatory factor, or in which cytokine regulatory factor expression can be induced to higher levels are therefore useful for the production of cytokines.

The methods of the invention rely on the use cells that overexpress a cytokine regulatory factor (a protein capable of regulating cytokine expression), exemplified herein by PKR, with no particular method of cytokine regulatory factor overexpression required. Additional cytokine regulatory factors for use in practicing the methods of the invention include, but are not limited to transcription factors such as IRF-7, IRF-3 and NF-KB. Overexpression of these factors may be used to increase the expression or production of cytokines.

Exemplary methods for obtaining a cell line that overexpresses or overproduces PKR include, but are not limited to, selecting for a cell that overexpresses PKR and growing that cell to produce a PKR overexpressing cell line and/or modifying a cell capable of producing PKR by introducing an exogenous PKR-encoding nucleic acid sequence (of heterologous or autologous origin) into the cell in a manner effective to express the coding sequence.

Selecting for a cell that overexpresses PKR can be accomplished by selection or subcloning of cells that overexpress PKR mRNA or protein, or that exhibit higher PKR kinase activity due to expression of the endogenous gene. Such subclones may be selected by choosing cells with higher functional PKR kinase activity, higher PKR mRNA levels or higher PKR protein levels than the corresponding parental cell line.

Although the mechanism is not part of the invention, enhanced PKR activity in these cell may be the result of (1) enhanced promoter activity leading to increased transcription; (2) PKR gene mutation resulting in a variant form of PKR with enhanced kinase activity; (3) multiple gene copies resulting in enhanced kinase activity; or (4) enhanced responsiveness of the promoter to stimuli including cellular stress signals (e.g., poly I:C, endotoxin, PMA, or heat shock).

PKR overexpressing cells can also be identified and selected by examining their functional ability to synthesize cytokines.

Once a cell line that overexpresses or overproduces PKR is obtained that cell line may be further primed or primed and treated in a manner effective to result in an increase in PKR and/or cytokine production.

In one preferred approach, the method comprises (a) culturing mammalian cells capable of PKR over-expression or an analog or homologue thereof under conditions sufficient to overexpress PKR; (b) priming; and (c) treating the cell culture as appropriate to induce the expression of a cytokine gene.

Similarly, exemplary methods for obtaining a cell line that overexpresses or overproduces a cytokine regulatory factor, such as an IFN regulatory factor (IRF) or other transcription factor include, but are not limited to, selecting for a cell that over expresses the factor, e.g., IRF-3 or IRF-7 and growing that cell to produce an IRF-3 or IRF-7 overexpressing cell line.

In an alternative approach, a cell capable of producing the factor may be modified by introducing a heterologous nucleic acid construct comprising the nucleic acid coding sequence for the factor, e.g., IRF-3 or IRF-7 (of heterologous or autologous origin), into the cell in a manner effective to express the coding sequence.

Cells that overexpress a cytokine regulatory factor, such as an IRF or other transcription factor may also be identified and selected by evaluating their functional ability to synthesize cytokines.

III. Enhanced Cytokine-Regulatory Factor Expression Is Associated With Enhanced Cytokine Production.

The invention is generally directed to the association between overexpression of a cytokine-regulatory factor and enhanced cytokine production.

By increasing the expression/activity of a cytokine regulatory factor, such as PKR in mammalian cells, cytokine production can be increased. Mammalian cell cultures that express a higher constitutive level of a cytokine regulatory factor, or in which cytokine regulatory factor expression can be induced to higher levels are therefore useful for the production of cytokines.

The method relies on the use of cells that overexpress a cytokine regulatory factor or a protein capable of regulating cytokine expression, exemplified herein by PKR, with no particular method of cytokine regulatory factor overexpression required.

Once a cell line that overexpresses or overproduces PKR is obtained that cell line may be further primed and treated in a manner effective to result in an increase in PKR and/or cytokine production.

In one preferred approach, the method comprises (a) culturing mammalian cells capable of overexpression of a cytokine regulatory factor; (b) introducing a heterologous nucleic acid construct comprising the coding sequence for the cytokine regulatory factor, or an analog or homologue thereof, under conditions sufficient to overexpress the cytokine regulatory factor; (c) priming; and (d) treating the cells as appropriate to induce the expression of a cytokine gene.

In one preferred embodiment, the cytokine-regulatory factor is PKR and the cells are inducible for PKR overexpression. In a preferred aspect, cells capable of overexpressing PKR are treated and/or induced in a manner, which results in a higher level of PKR expression or production.

In one preferred aspect of the invention, a combination of culture conditions, priming, priming and treating or inducing results in significantly enhanced cytokine production by a given cell line, e.g., an increase that represents at least 200% (2-fold or 2×), 250% (2.5-fold or 2.5×), 400% 3-fold or 3×), 400% (4-fold or 4×), 500% (5-fold or 5×), and preferably 1000% (10-fold or 2×), or more cytokine production or expression relative to the level exhibited by the same cell line under the same culture conditions absent one or more of treating, priming and inducing the cells as described herein. In some cases, the methods of the invention result in an increase in cytokine production that is 100-fold (100×) to 1000-fold (1000×) or more.

Accordingly, one aspect of the present invention pertains to an isolated population of cells, i.e. a cell line, which overexpresses or overproduces PKR. The term “population” as used herein refers to a group of two or more cells, which have been derived from a single parental cell.

Any of a number of known cell types are useful for making a PKR overexpressing cell line, with particular examples provided below.

Problems typically associated with production of cytokines in cell culture, for example low yield from non-recombinant mammalian systems, improper glycosylation, or misfolding of proteins produced in microbial systems are eliminated in the methods of the present invention, which should prove to be useful for the development of therapeutic proteins.

IV. Increasing Endogenous PKR Activity, Expression And/Or Production.

In one aspect, the invention provides a native cell line that overexpresses or overproduces an endogenous PKR coding sequence and methods of producing the same.

In accordance with the present invention, it has been discovered that cell lines capable of expressing PKR and one or more cytokines, may be subjected to limiting dilution cloning (referred to herein as “subcloning”), screened for enhanced PKR kinase activity and/or mRNA and/or protein expression, further subcloned and selected for enhanced cytokine activity and/or expression, as further detailed below. Exemplary cytokines, which may be used as markers for such enhanced cytokine regulatory factor overexpression, include but are not limited to TNF-beta, IL-6, IL-8 and IFNs.

In practicing the method, a cell line capable of expressing PKR and one or more cytokines (designated herein as the “parental cell line”) is identified and subjected to limiting dilution cloning of single cells, using standard methods routinely employed by those of skill in the art. In general, the subcloning step is carried out at least 3 times, preferably at least 5 times and typically from 5 to 10 times in 96 well plates. Subclones are grown to obtain a population of approximately 0.3 to 0.5 million cells/ml using culture conditions typically employed to culture the parental cell line. The subclones are then assayed for PKR expression by evaluating transcription (mRNA) and/or protein levels (Western blot) and/or kinase activity, using methods known in the art. (See, e.g., Gunnery, S. and Mathews, M. B., 1998.)

By way of example, assays for PKR activity include autophosphorylation assays (Der and Lau, Proc. Natl. Acad. Sci., 92:8841-8845, 1995), an assay for eIF2α phosphorylation (Zamanian-Daryoush, Der, Williams, Oncogenes, 18:315-326, 1999), and a kinase assay (carried out by immunoprecipitation of PKR and in vitro assay for kinase (Zamanian-Daryoush, et al, Molecular and Cellular Biology, 20:1278-1290, 2000).

Exemplary assays for PKR expression and/or production include protein assays such as Western blot and assays for PKR mRNA such as RT-PCR (reverse transcriptase polymerase chain reaction) and Northern blotting, dot blotting, or in situ hybridization using an appropriately labeled probe based on the PKR-encoding nucleic acid sequence.

Subclones that exhibit a level of PKR expression or production that is at least 2-fold (2×), and preferably 3-fold (3×), 4-fold (4×), 5-fold (5×) or more greater than the level of PKR expression or production of the parental cell line are selected. In some cases, such selected subclones exhibit a level of PKR expression or production that is 10-fold (10×) or more the level of PKR expression or production of the parental cell line. Selected subclones also exhibit a higher level of PKR expression and/or production. In one preferred approach, the level of PKR expression or production is determined by a PKR activity assay.

Selected subclones are then treated in a manner effective to result in enhanced PKR and cytokine activity, expression and/or production. Treating may include adding a microbial (e.g., viral) or non-microbial inducer to the cell culture, as further described below.

Typically, selected subclones are primed by exposure to a priming agent prior to being treated in a manner effective to result in enhanced PKR and cytokine production. Exemplary priming agents are also further described below.

V. Increasing PKR By Expression Of A Heterologous Nucleic Acid Construct In A Cell

The invention also provides host cells, which have been transduced, transformed or transfected with an expression vector comprising a PKR-encoding nucleic acid sequence. The culture conditions, such as temperature, pH and the like, are those previously used for the parental host cell prior to transduction, transformation or transfection and will be apparent to those skilled in the art.

In one approach, a mammalian cell line is transfected with an expression vector having a promoter or biologically active promoter fragment or one or more (e.g., a series) of enhancers which functions in the host cell line, operably linked to a DNA segment encoding PKR, such that the PKR is overexpressed in the cell line.

A. Vectors

Natural or synthetic polynucleotide fragments encoding PKR (“PKR-encoding nucleic acid sequences”) may be incorporated into heterologous nucleic acid constructs or vectors, capable of introduction into, and replication in, a mammalian cell. The vectors and methods disclosed herein are suitable for use in host cells for the expression of PKR. Any vector may be used as long as it is replicable and viable in the mammalian cells into which it is introduced. Large numbers of suitable vectors and promoters are known to those of skill in the art, and are commercially available. Appropriate cloning and expression vectors for use in human cells are also described in Sambrook, et al., 1989, and Ausubel F. M., et al., 1989, expressly incorporated by reference herein. The appropriate DNA sequence may be inserted into a plasmid or vector (collectively referred to herein as “vectors”) by a variety of procedures. In general, the DNA sequence is inserted into an appropriate restriction endonuclease site(s) by standard procedures. Such procedures and related sub-cloning procedures are deemed to be within the scope of knowledge of those skilled in the art.

Such vectors are typically equipped with selectable markers, insertion sites, and suitable control elements, such as termination sequences. The vector may comprise regulatory sequences, including, for example, non-coding sequences, such as introns and control elements, i.e., promoter and terminator elements or 5′ and/or 3′ untranslated regions, effective for expression of the coding sequence in host cells (and/or in a vector or host cell environment in which a modified soluble protein antigen coding sequence is not normally expressed), operably linked to the coding sequence. Large numbers of suitable vectors and promoters are known to those of skill in the art, are commercially available and are described in Sambrook, et al, (supra).

Exemplary promoters include both constitutive promoters and inducible promoters, examples of which include a CMV promoter, an SV40 early promoter, an RSV promoter, an EF-1α promoter, a promoter containing the tet responsive element (TRE) in the tet-on or tet-off system as described (ClonTech and BASF), the beta actin promoter and the metallothienein promoter that can upregulated by addition of certain metal salts.

The choice of the proper selectable marker will depend on the host cell, and appropriate markers for different hosts are well known in the art. Typical selectable marker genes encode proteins that (a) confer resistance to antibiotics or other toxins, e.g., ampicillin, methotrexate, tetracycline, neomycin (Southern and Berg, J., 1982), mycophenolic acid (Mulligan and Berg, 1980), puromycin, zeomycin, or hygromycin (Sugden et al., 1985).

In one preferred embodiment of the invention, PKR overexpression is achieved, using cells that comprise an exogenously provided PKR-encoding nucleic acid sequence, under the control of a suitable promoter, either constitutive or inducible, for PKR overexpression in cell culture.

B. PKR-Encoding Nucleic Acid Sequences

A vector comprising a PKR-encoding nucleic acid sequence may be introduced into a cell, resulting in overexpression of PKR by the cell. Exemplary coding sequences for use in such vectors include, but are not limited to the coding sequence from, the human p68 PKR gene found at GenBank Accession No. M35663, the murine PKR gene and other eIF-2-alpha kinases including yeast GCN2 and hemin regulated inhibitor (Wek, R. C., 1994).

In addition, mutants or variant forms of these PKR genes can be included in vector constructs for use in the overexpression of PKR. Upon expression, mutant or variant forms of these PKR proteins may have increased or decreased kinase activity.

In one approach, PKR mutant genes are generated using the transformer site-directed mutagenesis kit (ClonTech). Cai and Williams (1998) described a series of PKR mutants that exhibit dissociation of substrate binding (in terms of eIF-alpha) from kinase activity (phosphorylation of substrates). These mutant with their reduced PKR activity can be used for transfection of cells to generate PKR-expressing cells, albeit less effective in substrate binding or with less kinase activity.

A selected PKR coding sequence may be inserted into a suitable vector according to well-known recombinant techniques and used to transform a cell line capable of PKR overexpression, as further described below.

In accordance with the present invention, polynucleotide sequences which encode PKR, include splice variants, fragments of PKR, fusion proteins, modified forms or functional equivalents thereof, collectively referred to herein as “PKR-encoding nucleic acid sequences”. (See, e.g., Wu, S. and Kaufman, R. J., 1997.) Such “PKR-encoding nucleic acid sequences” may be used in recombinant DNA molecules (also termed heterologous nucleic acid constructs) that direct the expression of PKR in appropriate host cells.

Due to the inherent degeneracy of the genetic code, other nucleic acid sequences, which encode substantially the same or a functionally equivalent amino acid sequence may be used to clone and express PKR. Thus, for a given PKR-encoding nucleic acid sequence, it is appreciated that as a result of the degeneracy of the genetic code, a number of coding sequences can be produced that encode the same PKR amino acid sequence. For example, the triplet CGT encodes the amino acid arginine. Arginine is alternatively encoded by CGA, CGC, CGG, AGA, and AGG. Therefore it is appreciated that such substitutions in the coding region fall within the sequence variants covered by the present invention. Any and all of these sequence variants can be utilized in the same way as described herein for a parent PKR-encoding nucleic acid sequence.

A “variant” PKR-encoding nucleic acid sequence may encode a “variant” PKR amino acid sequence, which is altered by one or more amino acids from the native PKR polypeptide sequence, both of which are included within the scope of the invention. Similarly, the term “modified form of”, relative to PKR, means a derivative or variant form of the native PKR-encoding nucleic acid sequence or protein. That is, a “modified form of” PKR has a derivative sequence containing at least one nucleic acid or amino acid substitution, deletion or insertion. The nucleic acid or amino acid substitution, insertion or deletion may occur at any residue within the sequence, as long as the encoded amino acid sequence maintains the biological activity of the native PKR protein, e.g., autophosphorylation activity or eukaryotic translation initiation factor 2 (eIF-2) phosphorylation activity.

A “variant” PKR-encoding nucleic acid sequence may encode a “variant” PKR amino acid sequence, which contains amino acid insertions or deletions, or both. Furthermore, a variant PKR coding sequence may encode the same polypeptide as the reference polynucleotide or native sequence but, due to the degeneracy of the genetic code, has a nucleic acid coding sequence that is altered by one or more bases from the reference or native polynucleotide sequence.

The variant nucleic acid coding sequence may encode a variant amino acid sequence which contains a “conservative” substitution, wherein the substituted amino acid has structural or chemical properties similar to the amino acid which it replaces and physicochemical amino acid side chain properties and high substitution frequencies in homologous proteins found in nature (as determined, e.g., by a standard Dayhoff frequency exchange matrix or BLOSUM matrix). In addition, or alternatively, the variant nucleic acid coding sequence may encode a variant amino acid sequence that contains a “non-conservative” substitution, wherein the substituted amino acid has dissimilar structural or chemical properties to the amino acid that it replaces. Standard substitution classes include six classes of amino acids based on common side chain properties and highest frequency of substitution in homologous proteins in nature, as is generally known to those of skill in the art and may be employed to develop variant PKR-encoding nucleic acid sequences.

PKR-encoding nucleotide sequences also include “allelic variants” defined as an alternate form of a polynucleotide sequence that may have a substitution, deletion or addition of one or more nucleotides, which does not substantially alter the function of the encoded polypeptide.

The polynucleotides for use in practicing the invention include sequences that encode PKR and PKR splice variants, sequences complementary to the protein coding sequence, and novel fragments of the polynucleotide. The polynucleotides may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

As will be understood by those of skill in the art, in some cases it may be advantageous to produce PKR-encoding nucleotide sequences possessing non-naturally occurring codons. Codons preferred by a particular eukaryotic host (Murray, E., et al., 1989) can be selected, for example, to increase the rate of PKR polypeptide expression or to produce recombinant RNA transcripts having desirable properties, such as a longer half-life, than transcripts produced from naturally occurring sequence.

In one general embodiment, a PKR-encoding nucleotide sequence has at least 70%, preferably 80%, 85%, 90% or 95% or more sequence identity to the human p68 PKR coding sequence found at GenBank Accession No. M35663, expressly incorporated by reference herein.

A PKR-encoding nucleotide sequence may be engineered in order to alter the PKR coding sequence for a variety of reasons, including but not limited to, alterations, which modify the cloning, processing and/or expression of PKR by a cell.

Heterologous nucleic acid constructs may include the coding sequence for PKR, a variant, fragment or splice variant thereof: (i) in isolation; (ii) in combination with additional coding sequences; such as fusion protein or signal peptide, in which the PKR coding sequence is the dominant coding sequence; (iii) in combination with non-coding sequences, such as introns and control elements, such as promoter and terminator elements or 5′ and/or 3′ untranslated regions, effective for expression of the coding sequence in a suitable host; and/or (iv) in a vector or host environment in which the PKR coding sequence is a heterologous gene.

The present invention also includes recombinant nucleic acid constructs comprising one or more of the PKR-encoding nucleic acid sequences as described above. The constructs comprise a vector, such as a plasmid or viral vector, into which a sequence of the invention has been inserted, in a forward or reverse orientation.

C. Selection and Transformation of Host Cells

A vector containing the appropriate nucleic acid coding sequence, as described above, together with appropriate promoter and control sequences, may be employed to transform a mammalian cell to permit the cells to overexpress PKR and thereby enhance cytokine expression or production.

In one aspect of the present invention, a heterologous nucleic acid construct is employed to transfer a PKR-encoding nucleic acid sequence into a cell in vitro, with established cell lines preferred. For long-term, high-yield production of cytokines, stable expression is also preferred. It follows that any method effective to generate stable transformants may be used in practicing the invention.

The practice of the present invention will employ, unless otherwise indicated, conventional techniques of molecular biology, microbiology, recombinant DNA, and immunology, which are within the skill of the art. Such techniques are explained fully in the literature. See, e.g., MOLECULAR CLONING: A LABORATORY MANUAL, Third Edition (Sambrook, Russell & Sambrook, eds., 2001), ANIMAL CELL CULTURE (R. I. Freshney, ed., 1988); CURRENT PROTOCOLS IN MOLECULAR BIOLOGY (F. M. Ausubel et al., eds., John Wiley & Sons, 1993); and CURRENT PROTOCOLS IN IMMUNOLOGY (J. E. Coligan et al., eds., John Wiley & Son, 1994). All patents, patent applications, articles and publications mentioned herein, both supra and infra, are hereby expressly incorporated herein by reference.

The invention provides cells and cell compositions which have been genetically modified to comprise an exogenously provided PKR-encoding nucleic acid sequence. A parental cell or cell line may be genetically modified (i.e., transduced, transformed or transfected) with a cloning vector or an expression vector. The vector may be, for example, in the form of a plasmid, a viral particle, a phage, etc., as further described above.

Various methods may be employed for delivering a vector into cells in vitro. Heterologous nucleic acid constructs comprising a PKR-encoding nucleic acid sequence can be transcribed in vitro, and the resulting RNA introduced into the host cell by well-known methods, e.g., by injection. Methods of introducing nucleic acids into cells for expression of heterologous nucleic acid sequences are also known to the ordinarily skilled artisan, including, but not limited to electroporation; nuclear microinjection or direct microinjection into single cells; bacterial protoplast fusion with intact cells; use of polycations, e.g., polybrene or polyornithine; membrane fusion with liposomes, lipofectamine or lipofection-mediated transfection; high velocity bombardment with DNA-coated microprojectiles; incubation with calcium phosphate-DNA precipitate; DEAE-Dextran mediated transfection; infection with modified viral nucleic acids; and the like. (See, e.g., Davis, L., Dibner, M., and Battey, I., BASIC METHODS IN MOLECULAR BIOLOGY, 1986.)

The genetically modified cells can be cultured in conventional nutrient media modified as appropriate for activating promoters, selecting transformants or amplifying expression of a PKR-encoding nucleic acid sequence. The culture conditions, such as temperature, pH and the like, are those previously used for the host cell selected for expression, and will be apparent to those skilled in the art.

The progeny of cells into which such heterologous nucleic acid constructs have been introduced are generally considered to comprise the PKR-encoding nucleic acid sequence found in the heterologous nucleic acid construct.

D. Cells and Culture Conditions For Enhanced PKR Expression.

Thus, the present invention provides a cell line comprising cells, which have been selected, modified, primed and/or primed and treated in a manner effective to result in enhanced PKR production or expression relative to the corresponding parental cell line.

Examples of parental cell lines which may be treated and/or modified for enhanced PKR expression include, but are not limited to B cells (Namalwa, 293, Raji), monocytic cells (U937, THP-1) , Vero, MRC-5, WI-38 cells, Flow 1000 cells, Flow 4000 cells, FS-4 and FS-7 cells, fibroblasts (MRC-5, MG-63 cells), CCRF-SB cells, T cells (CCRF-CEM, Jurkat) cells and T98G cells. Examples of appropriate primary cell types which may selected, modified, primed and/or primed and treated for enhanced PKR expression include, but are not limited to, cells of the monocyte/macrophage lineage, lymphocytic lineage cells including T- and B-cells, mast cells, fibroblasts, bone marrow cells, keratinocytes, osteoblast derived cells, melanocytes, endothelial cells, platelets, various other immune system cells, lung epithelial cells, pancreatic parenchmal cells, glial cells and tumor cells derived from such cell types. PKR-over expressing cells are cultured under conditions typically employed to culture the parental cell line.

Generally, cells are cultured in a standard medium containing physiological salts and nutrients, such as standard RPMI, MEM, IMEM or DMEM, typically supplemented with 5-10% serum, such as fetal bovine serum. Culture conditions are also standard, e.g., cultures are incubated at 37° C. in stationary or roller cultures until desired levels of PKR expression are achieved.

Preferred culture conditions for a given cell line may be found in the scientific literature and/or from the source of the cell line such as the American Type Culture Collection (ATCC; “http://www.atcc.org/”). Preferred culture conditions for primary cell lines, such as fibroblasts, B-cells, T-cells, endothelial cells, dendritic cells, and monocytes are generally available in the scientific literature. After cell growth has been established, the cells are exposed to conditions effective to cause or permit the overexpression of PKR.

In cases where the PKR gene is under the control of an inducible promoter, the inducing agent, e.g., a metal salt or antibiotics, is added to the medium at a concentration effective to induce high-level PKR expression.

E. Treatment Of Cells To Further Enhance PKR Expression.

In general, additional steps are taken to enhance expression of PKR and one or more cytokines, or facilitate recovery of cytokines from mammalian cell lines. Such steps include one or more of (1) culturing the cells under conditions effective to enhance expression of PKR; (2) priming the PKR-expressing cells with reagents including, but not limited to polypeptides, chemicals, or nucleic acids; and (3) treating the PKR-expressing cells to induce production of PKR and one or more cytokines (induction).

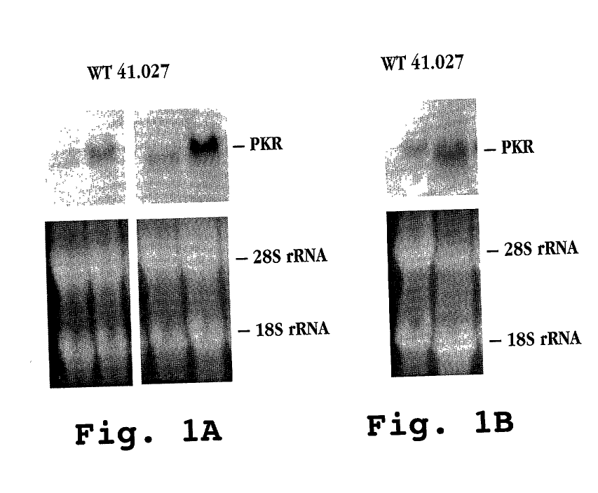

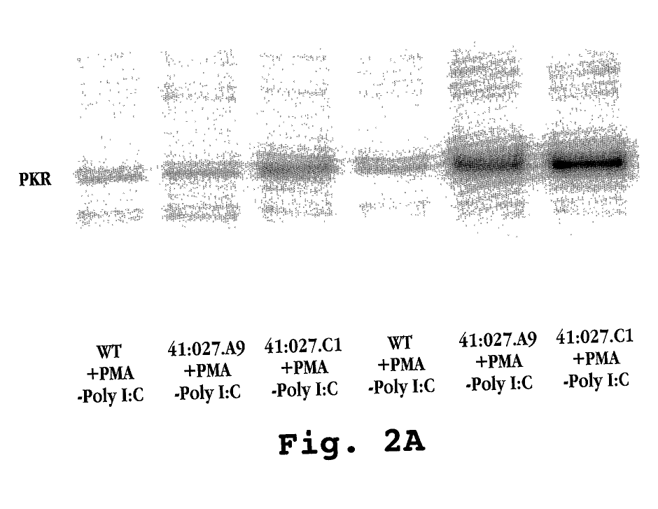

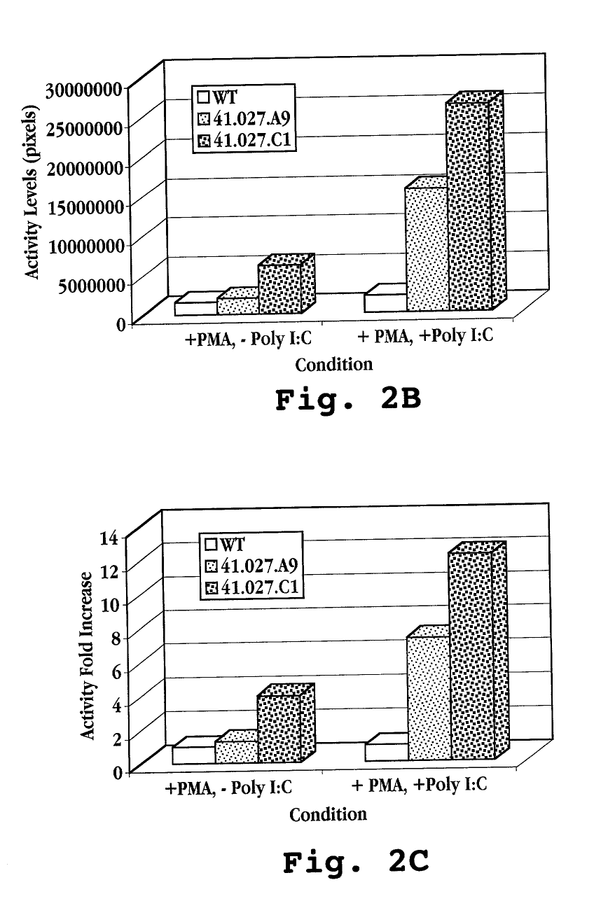

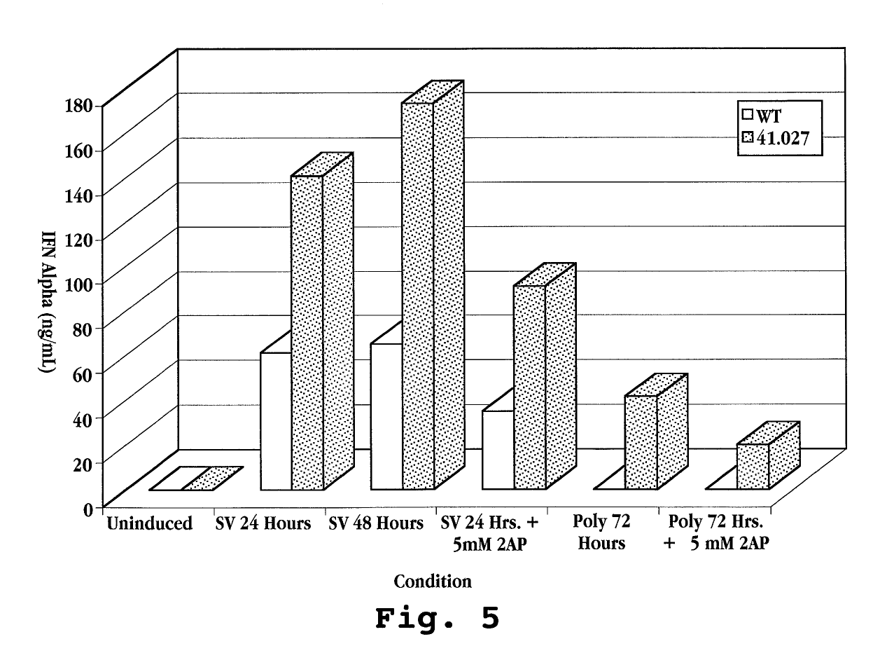

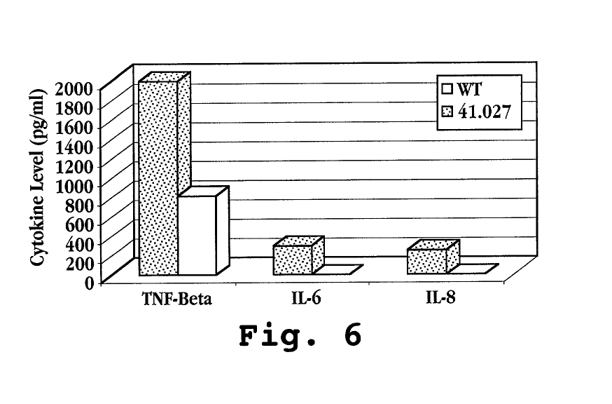

In one exemplary approach, treatment of PKR overexpressing cell lines (41.027.A9 and 41.027.C1) with poly I:C was effective to induce greater PKR expression than observed for wild type Namalwa-cells (FIG. 2B). In addition, the results provided herein demonstrate that a PKR overexpressing cell line derived by subcloning and selection, such as a 41.027.A9 and 4.027.C1 clone and its subcloned progeny cell lines, collectively are more responsive to induction of PKR activity and expression (FIGS. 1, 2 and 3) than a corresponding wild type cell line. Such a PKR overexpressing cell line thus produces higher levels of cytokines, as exemplified for IFN in FIG. 5 and for TNF-beta, IL-6 and IL-8 in FIG. 6.

Culturing the cells under conditions effective to facilitate recovery of cytokines include, but are not limited to culture in serum and/or protein-free or serum-free medium.

Priming is a well known phenomenon whereby pretreatment of cells with a priming agent results in enhanced production of one or more cytokines, following subsequent treatment or induction. Exemplary priming agents include, but are not limited to phorbol myristate acetate (PMA) and other phorbol esters, calcium ionophores, interferon-α, interferon-γ, interferon-β, G-CSF, GM-CSF, PDGF, TGF, EGF or chemokines (IL-8, MCP or MIP), sodium butyrate, endotoxin, a kinase activator (e.g., protein activator of PKR, PACT), or a transcription activator (NF-KB, IRFs including IRF-3 and IRF-7). Suppression of a PKR inhibitor, p53, has also been demonstrated to result in enhanced PKR activity (Tan, et al., 1998). Alternatively, deprivation of serum and growth factors such as IL-3 may be used to induce PKR activity in cells. Suitable priming agent concentrations may be found in the scientific literature, e.g., a concentration of PMA in the range 5-50 nM, preferably about 10 nM, is suitable.

Treating may include adding a microbial, (viral, bacterial, or fungal) inducer, an extract of a microbe capable of acting as an inducer (e.g., an endotoxin or bacterial cell wall containing extract) or a non-microbial inducer to the cell culture. Exemplary non-microbial include, but are not limited to, double-stranded RNA (dsRNA) such as poly(l):poly(C) (poly I:C), small molecules, e.g., polyanions, heparin dextran sulfate, chondroitin sulfate and cytokines.

Exemplary methods of viral induction include, but are not limited to, (1) exposure to live virus (such as Sendai virus, encephalomyocarditis virus or Herpes simplex virus); (2) exposure to the aforementioned killed virus; or (3) exposure to isolated double-stranded viral RNA. In addition, cytokine induction may be produced or enhanced by adding particular cytokines known to stimulate cytokine production in certain cells.

After addition of the inducing agent, typically cells are further incubated until desired levels of induced and secreted cytokines are obtained. Incubation at 37° C. for at least 12-48 hours, and up to 72-96 hours is generally sufficient.

In one preferred approach, overexpression of PKR and one or more cytokines is effected by further treatment of the cells with DEAE Dextran.

The nucleoside analog, 2-aminopurine (2-AP) is a known inhibitor of PKR. FIG. 5 illustrates inhibition of PKR activity with consequent suppression of cytokine induction by treatment with 2-AP. 2AP is typically used at a concentration of about 0.5 to 5 mM.

In another approach, PKR expression may be enhanced by a regulatory factor, which interacts with the promoter controlling the expression of a PKR-encoding nucleic acid sequence. In the case of expression of the endogenous PKR-encoding nucleic acid sequence, exemplary regulatory factors include the interferon-inducible GAS elements, the IL-6 sensitive NF-IL6 and APRF elements and NF-κB elements. (See, e.g., Jagus R. et al., 1999 and Williams, B. R., 1999.)

VI. Methods Of Evaluating PKR Expression

The activity, expression and/or production of a given cytokine-regulatory factor can be determined by methods known in the art.

By way of example, the presence, amplification and/or expression of an endogenous or exogenously provided PKR-encoding nucleic acid sequence may be measured in a sample directly, for example, by assays for PKR activity, expression and/or production. Such assays include autophosphorylation assays, an assay for eIF2α phosphorylation, a kinase assay; Northern blotting to quantitate the transcription of mRNA, dot blotting (DNA or RNA analysis), RT-PCR (reverse transcriptase polymerase chain reaction), or in situ hybridization, using an appropriately labeled probe, based on the PKR-encoding nucleic acid sequence; and conventional Southern blotting.

Alternatively, gene expression, may be measured by immunological methods, such as immunohistochemical staining of cells or tissue sections to directly evaluate expression of PKR, Western blot or ELISA. Antibodies useful for immunohistochemical staining and/or assay of sample fluids may be either monoclonal or polyclonal, and may be prepared in any mammal.

The details of such methods are known to those of skill in the art and many reagents for practicing such methods are commercially available.

VII. Additional Factors Effective To Enhance Cytokine Production

Apoptosis or programmed cell death is a cell-intrinsic suicide process whereby unwanted individual cells undergo a genetically determined program, culminating in chromosomal DNA fragmentation, degradation of RNA and eventual cell death (reviewed in Orrenius 1995; Stellar 1995; Vaux 1993). Once committed to apoptosis, the cells undergo new rounds of protein synthesis and various morphological/physiological changes including cytoplasmic condensation, nuclear chromatin condensation, membrane blebbing, and eventual DNA degradation, detected as a characteristic oligonucleosomal ladder.

TNFs, as prototypes proinflammatory cytokines, are cytotoxic proteins produced by activated immune cells during the processes of pathogen elimination, antiviral activities, and tumor destruction. However, high levels of TNF-alpha in vivo can be detrimental since TNF-alpha induces metabolic disturbances, wasting, and suppression of hematopoiesis. At the cellular level, TNF-alpha induces production of superoxide radicals, activation of lysosomal enzymes (Larrick, et al., 1990; Liddil, et al., 1989), and fragmentation of DNA by the activation of endonuclease activity (Rubin, et al., 1988), leading to apoptosis.

PKR is known to play a role in the TNF-α signaling pathway and in the induction of apoptosis. It has been shown that U937 cells, which over express PKR, also exhibit increased apoptosis. (See, e.g., Yeung, M. C., et al., 1996 and 1999.)

Accordingly, in one aspect of the present invention, a PKR-overexpressing, cytokine-producing cell line, is treated to inhibit apoptosis.

For example, cells are transfected with a gene encoding a protein that is capable of inhibiting apoptosis in the cells, under the control of a suitable promoter.

Typically, the cells are first transfected with a vector containing an anti-apoptotic gene, then successful transformants are further transfected with a vector containing a cytokine regulatory factor gene. This allows for the second transfection and selection to be carried out with cells that have already been “stabilized” with an anti-apoptopic function. Alternatively, a subcloned and selected PKR overexpressing cell is grown to generate a PKR overexpressing cell line into which is introduced a vector containing an anti-apoptotic gene.

Methods for enhancing the production of cytokines in cell culture by inhibiting apoptosis associated with cytokine synthesis, particularly under conditions of PKR overexpression are further described in co-owned U.S. Provisional Application No. 09/657,881, expressly incorporated by reference herein.

VIII. Cytokine Production

Cytokines produced by the cells that overexpress PKR are secreted into the medium and may be purified or isolated, e.g., by removing unwanted components from the cell culture medium. In general, the cytokines are fractionated to segregate cytokines having selected properties, such as binding affinity to particular binding agents, e.g., antibodies or receptors; or which have a selected molecular weight range, or range of isoelectric points.

A. Cytokines Overexpressed In Conjunction With PKR Overexpression

Exemplary cytokines the expression of which may be increased using the methods of the invention include, but are not limited to, interferons (α, β and γ), interleukins (IL-1α, IL-1β, IL-1ra, IL-2 and IL-4 through 13), tumor necrosis factors alpha and beta (TNF-β) and their respective soluble receptors (sTNF-R), the colony stimulating factors (granulocyte colony stimulating factor, G-CSF; granulocyte-macrophage colony stimulating factor, GM-CSF; and IL-3), the angiogenic factors (fibroblast growth factor, FGF; vascular endothelial growth factor, VEGF; and platelet-derived growth factors 1 and 2 (PDGF-1 and -2) and the anti-angiogenic factors (angiostatin and endostatin).

In the case where the cytokine regulatory factor, e.g., PKR, overexpressing cell line is produced by recombinant DNA technology, the cytokine regulatory factor may be expressed under the control of an inducible promoter, e.g., a metallothionein promoter or a tetracycline (TRE) promoter (tet-on or tet-off); a constitutive promoter, e.g., a CMV promoter or an SV-40 promoter.

Production of cytokines by cells transfected with a vector encoding PKR has been previously described for interferon in co-owned U.S. application Ser. No. 09/444,224 (allowed) and for other cytokines, alone or in combination, e.g., IL-1-alpha, IL-1-beta, IL-2 , IL-3, IL-4, IL-5, IL-6, IL-7, IL-8, IL-9, IL-10, IL-11, IL-12, IL-13 and TGF-beta in co-owned U.S. application Ser. Nos. 09/595,338 and 09/660,468, all of which are expressly incorporated by reference herein.

B. Combinations Of Cytokines.

The methods described herein find further utility in the production of a mixture of two or more cytokines for therapeutic uses, and in particular, a cytokine mixture for use in treating cancer, viral infection, or inflammation, as detailed in co-owned U.S. Provisional Application No. 09/660,468, expressly incorporated by reference herein.

In summary, a PKR-overexpressing cell line is cultured under appropriate conditions, induced and/or treated in a manner effective to result in production of two or more cytokines and cytokines produced by the cells and secreted into the medium are isolated, e.g., removing unwanted components from the cell culture medium.

The composition is preferably produced by fractionating the collected cytokines to isolate cytokines having selected properties, such as binding affinity to binding agents such, as antibodies or receptors, or a selected molecular weight range, or range of isoelectric points.

C. Purification and/or Isolation of Cytokines.

In one approach, to simplify the isolation of cytokines from cells that over express a cytokine regulatory factor, e.g., PKR, the cells are cultured in serum-containing medium, but the inducing and isolating steps are typically carried out in medium that is substantially serum and/or protein free.

Once increased expression of a given cytokine is achieved, the cytokine is isolated and/or purified from the cell culture. The isolating step may include contacting culture medium containing secreted cytokines with a solid support having surface-attached antibodies specific against the cytokine(s) to be isolated, washing the solid support to remove non-bound material, and eluting the cytokines specifically bound to the support.

Exemplary procedures suitable for such isolation and/or purification include the following: antibody-affinity column chromatography, ion exchange chromatography; ethanol precipitation; reverse phase HPLC; chromatography on silica or on a cation-exchange resin such as DEAE; chromatofocusing; SDS-PAGE; ammonium sulfate precipitation; and gel filtration using, for example, Sephadex G-75. Various methods of protein purification may be employed and such methods are known in the art and described for example in Deutscher, METHODS IN ENZYMOLOGY, 182, 1990; Scopes, PROTEIN PURIFICATION: PRINCIPLES AND PRACTICE, Third Edition, Springer-Verlag, New York, 1994. The purification step(s) selected will depend, for example, on the nature of the production process used and the particular cytokine produced.

At various time points following induction of cytokines, the cell medium may be tested for the presence or one or more selected cytokines. The presence of selected cytokines may be assayed by direct detection, e.g., with an antibody binding assay (see below) or indirectly by the effect of the culture medium on the activity of various cytokine-responsive cells, according to biological assays typically employed in the art.

D. Methods Of Evaluating Cytokine Expression

The activity, expression and/or production of a given cytokine may also be determined by methods known in the art. Examples include Northern blot and reverse transcriptase polymerase chain reaction (RT-PCR) for mRNA. In addition, immunoassays, such as ELISA, competitive immunoassays, radioimmunoassays, Western blot, indirect immunofluorescent assays and the like may be used to detect the expressed protein.

In general, kits for cytokine analysis are commercially available and may be used for the quantitative immunoassay of the expression level of known cytokines or other proteins (e.g., cytokine detection kits available from R&D Systems).

In one preferred aspect of the invention, a combination of culture conditions, treating, priming and inducing results in significantly enhanced cytokine production by a given cell line, e.g., an increase that represents at least 2-fold (2×), 2.5-fold (2.5×), 3-fold (3×), 4-fold (4×), 5-fold (2×), and preferably 10-fold (10×) or more cytokine production or expression relative to the level exhibited by the same cell line under the same culture conditions absent one or more of treating, priming and inducing the cells as described herein. In some cases, the methods of the invention result in an increase in cytokine production that is 100-fold (100×) to 1000-fold (1000×) or more.

All patent and literature references cited in the present specification are hereby expressly incorporated by reference in their entirety.

The following examples illustrate but are not intended in any way to limit the invention.

### EXAMPLE 1

### Preparation Of PKR Over Expressing Cell Lines

Wild type Namalwa cells were obtained from the American Type Culture Collection (ATCC), 10801 University Blvd., Manassas, Va., U.S.A, These cells were cultured, subcloned, selected, primed and induced, as detailed below.

Wild type Namalwa cells were cultured in DMEM/F12 medium containing fetal bovine serum with a range of concentrations from 0.5 to 15%. The Namalwa cells were subjected to limiting dilution cloning (referred to herein as “subcloning”), by culture in 96 well plates, using standard methods routinely employed by those of skill in the art. The subcloning step yields clones arising as the progeny of a single cell. The subcloning was carried out from 5 to 10 times and subclones were grown to obtain a population of approximately 0.3 to 0.5 million cells/ml using culture conditions typically employed to culture the parental cell line. Subclones were then assayed for PKR expression and activity by Northern blot, Western blot, autophosphorylation assay (Der and Lau, Proc. Natl. Acad. Sci. 1995; 92: 8841-8845), an assay for eIF2α phosphorylation (Zamanian-Daryoush, et al.), and a kinase assay (carried out by immunoprecipitation of PKR combined with an in vitro assay for kinase (Zamanian-Daryoush, et al., 2000).

Subclones that exhibited at least 2-fold more kinase activity than the parental cell line were selected. Selected subclones were also screened for PKR production and/or expression by Western and Northern blot. Selected subclones were then treated to induce enhanced PKR and cytokine activity, production and/or expression, with or without priming prior to induction.

Cell lines were generated which expressed 2-fold or greater levels of PKR activity than wild type Namalwa cells in the absence of priming or induction. An exemplary PKR overexpressing cell line designated Namalwa PKR++41.027 cells, is used herein to illustrate the invention.

### EXAMPLE 2

### Characterization Of PKR Over Expressing Cell Lines

A. Enhanced Endogenous PKR Expression In Response To Non-Viral Induction

Cells from individual subclones of Namalwa PKR++41.027 cells and WT Namalwa cells were pretreated with 20 nM phorbol myristate acetate (PMA) at a density 5.0×10⁵ cells/ml in T150 flasks, for 24 h. This was followed by treatment with 200 μg/ml poly I:C and 10 μg/ml DEAE Dextran for an additional 48 hrs to induce PKR expression. Total RNA was isolated by using the single-step acid guanidinium thiocyanate-phenol-chloroform method described in Chomczynski P., et al., (1987).

Northern blots were prepared using 20 μg of total RNA isolated as described above, electrophoresed under denaturing conditions on 1% agarose gel containing 2.2M formaldehyde, and then transferred onto Protran membrane (Schleicher & Schuel). A PKR-Eco R1 fragment was used as a specific probe for hybridization. Filters were prehybridized for 5 hrs with standard hybridization solution containing 40% formamide and then hybridized overnight with ³²P labeled probe (labeled by a random priming reaction using a commercial labeling kit, obtained from Amersham). Membranes were then washed 2 times for 15 min at 60° C. with 2×SSC (0.3M NaCl, 0.03M sodium citrate, pH 7), 0.1% SDS and once at the same temperature with 0.1×SSC, 0.1% SDS.

FIG. 1A illustrates enhanced PKR expression following poly I:C induction in Namalwa PKR++41.027 cells relative to PKR expression in wild type Namalwa cells. The top panel shows a northern blot analysis of RNA isolated from resting cells (Lanes 1 and 3) and cells treated with poly I:C as described above (Lanes 2 and 4). A ³²P-labeled PKR EcoR1 fragment was used as the probe. As a control, the bottom panel presents pictures of the gel stained with ethidium bromide. A comparison of the intensity of the rRNA bands shows that the total amount of RNA loaded in each lane is similar. The size of 28S rRNA is ˜5 kb and that of 18S rRNA is ˜1.9 kb. Thus the increases in PKR band intensity in the top panels is due to increased levels of PKR RNA and not due to differences in the amount of total RNA present in each lane.

B. Enhanced Endogenous PKR Expression In Response To Viral Induction

Cells from individual subclones of Namalwa PKR++41.027 cells and WT Namalwa cells were pretreated with 1 mM sodium butyrate for 24 hrs. at 37° C. at a density 5.0×10⁵ cells/ml in T150 flasks. This was followed by treatment with 500 HAU/ml of Sendai virus (SV) for an additional 24 hrs. RNA was isolated by the using single-step acid guanidinium thiocyanate-phenol-chloroform method as described (P. Chomozynski et al., 1987).

Northern analysis was carried out using 20 μg of total RNA which was electrophoresed under denaturing conditions on 1% agarose gels containing 2.2 M formaldehyde followed by transfer onto Protran membranes (Schleicher & Schuel). A PKR-Eco R1 fragment was used as a specific probe for hybridization using a commercial BrightStar nonisotopic detection kit (Ambion).

FIG. 1B illustrates enhanced PKR expression following Sendai virus induction in Namalwa PKR++41.027 cells (Lane 2) relative to PKR expression in wild type (WT) Namalwa cells (Lane 1). As a control, the bottom panel presents pictures of the gel stained with ethidium bromide. A comparison of the intensity of the rRNA bands shows that the total amount of RNA loaded in each lane is similar. The size of 28S rRNA is ˜5 kb and that of 18S rRNA is ˜1.9 kb. Thus the increases in PKR band intensity in the top panels is due to increased levels of PKR RNA and not due to differences in the amount of total RNA present in each lane.

As can be seen from the results presented in FIGS. 1A and 1B, following priming and non-viral or viral induction, a PKR overexpressing cell line exemplified herein by Namalwa 41.027 cells can be induced to produce significantly more PKR than wild type Namalwa cells subjected to the same treatment. The results can be quantified by laser densitometry.

C. Kinase Assay of Uninduced Versus polyI:C-induced WT and 41.027 Cell Lines

Namalwa WT and subcloned progeny cell lines 41.027.A9 , and 41.027.C1 were cultured in DMEM/F12 medium containing 10% fetal bovine serum and 2 mM L-glutamine and were incubated at 37° C. in the presence of 5% CO₂. Cells were harvested, counted using a trypan blue dye exclusion assay and seeded into cell culture-treated flasks at a final cell density of 50×10⁵ cells/mL. Duplicate flasks were prepared for each cell line. The cells were primed by treating with 20 nM phorbal myristate acetate (PMA) for 20 hr. Following exposure to PMA, one set of flasks for each cell line was then induced by treatment with a final concentration of 200 μg/mL poly I:C plus 10 μg/mL DEAE Dextran for 48 hr.

Following the priming and induction steps, cell extracts were prepared, PKR was immunoprecipitated and PKR kinase activity was determined by an in vitro assay (Zamanian-Daryoush, et al., 2000). For this purpose, cells were washed twice in PBS and the cell pellet was resuspended in 60 to 90 μl of immunoprecipitation (IP) lysis buffer as described. The cell lysate was incubated on ice for 20 min followed by centrifugation at 20,800 g, for 20 min at 4° C. PKR was immunoprecipated and subjected to an in vitro kinase assay (Zamanian-Daryoush, et al., 1999). Briefly, PKR was immunoprecipitated from 100 μg of total lysate protein using an anti-PKR monoclonal antibody (MAb 71/10 obtained from Ara Hovanssian of the Institute Pasteur). After a 30 min incubation on ice, Protein G Sepharose beads were added for an 24 hr incubation at 4° C., the bead-bound kinase was washed twice with lysis buffer and followed by washing with DBGA (10 mM Tris-HCl, pH 7.6, 50 mM KCl, 2 mM magnesium acetate, 20% (v/v) glycerol, 7 mM mercaptoethanol). Immunoprecipitated PKR was incubated for 20 min at 30° C. in DBGA containing 0.83 mM manganese chloride, 0.8 μM ATP, 10 μ of purified histone, and 9 μCi of γ[³²P]ATP. The kinase reaction was terminated by the addition of 4×SDS-PAGE sample buffer. The phosphorylated PKR proteins were resolved on gradient (4-12% polyacrylamide) SDS-PAGE gels. The phosphorylated PKR proteins were then transferred and immobilized onto PVDF membranes utilizing standard protocols well known in prior art. The amount of radioactivity contained within the PKR band was quantitated using the phosphoimager capability of a Molecular Dynamics Typhoon 8600 Variable Mode Analyzer, coupled with ImageQuant Software vs 5.2.

FIG. 2A shows the autoradiographic image of SDS-PAGE PKR autokinase activity assay. The assay was conducted with Namalwa-WT and Namalwa-PKR++subcloned cell lines after exposure to PMA alone or to PMA and Poly I:C/DEAE Dextran as described above. Lanes 1-3 present samples which were primed with PMA for 20 hrs. but were not induced with Poly I:C/DEAE Dextran; Lanes 4-6 present samples which were primed with PMA for 20 hrs. followed by induction with Poly I:C and DEAE Dextran for 48 hr.

The PKR kinase activities were quantitated and the results are presented in FIGS. 2B and 2C. As shown, the subcloned cell lines (Namalwa-PKR++41.027.A9 and 41.027.C1) are more responsive to poly I:C treatment with a greater than two-fold increase of PKR kinase activity over the WT cell line following induction.

D. Western Blot Analysis of Uninduced Versus poly I:C induced WT and 41.027 cell lines

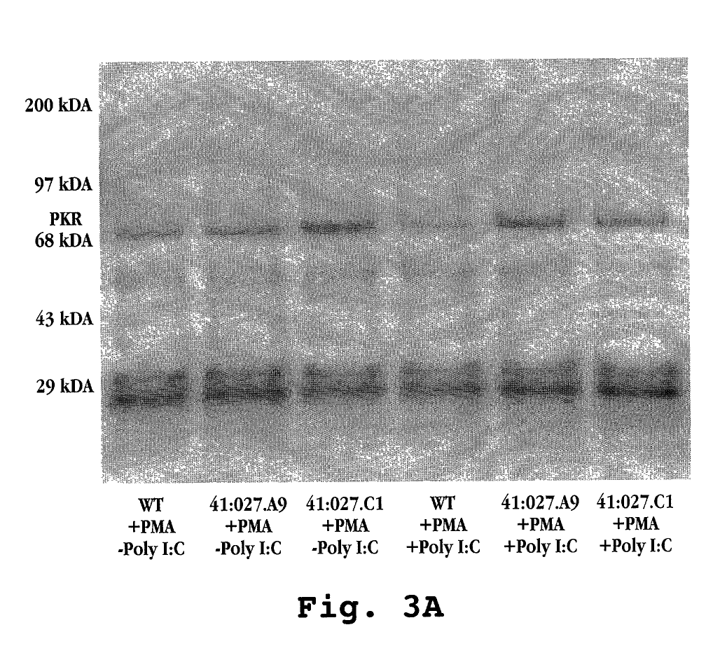

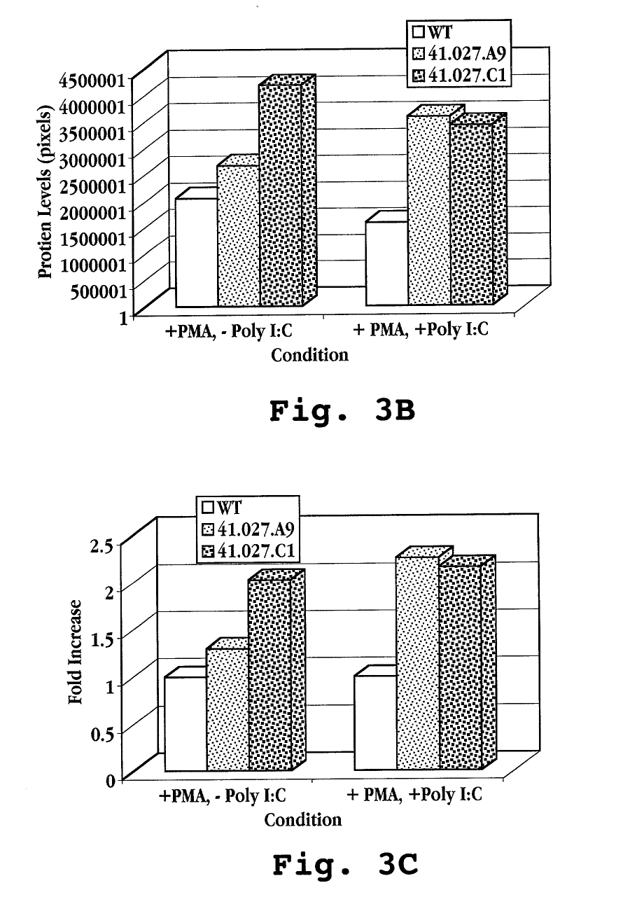

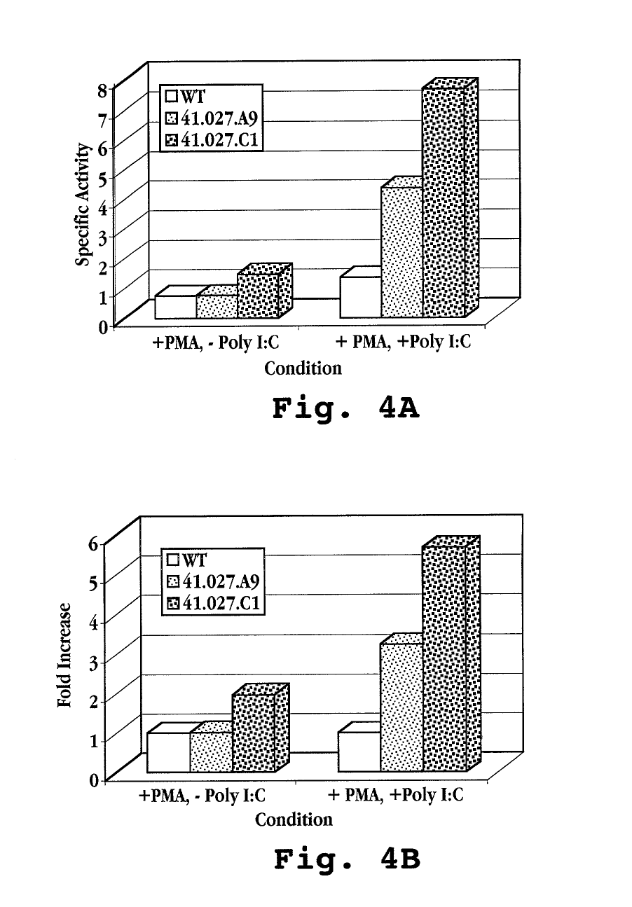

After the PKR proteins were immobilized onto the PVDF membrane and evaluated for PKR kinase activity as outlined in section C, the same membrane was further processed to evaluate the levels of total PKR protein using enzyme-amplified chemifluorescence Western Blot analysis. For this purpose, the membranes were incubated with a rabbit polyclonal anti-PKR antibody (obtained from Santa Cruz Biotechnology, Inc., Santa Cruz, Calif.) as a primary antibody, followed by detection with an anti-rabbit fluorescein-conjugated polyclonal antibody as the secondary antibody; and then an anti-fluorescein alkaline phosphatase-conjugated tertiary antibody. Signal was developed by the addition of the fluorogenic substrate ECF (Amersham). The Western blots were evaluated using the image analysis capability of the Typhoon 8600 Variable Mode Analyzer (Molecular Dynamics). The ECF reaction was detected using a laser with an excitation wave length of 532 nm, the photo multiplier tube was set at 500 volts with the instrument resolution set at 100 pixels with medium sensitivity. The data was analyzed using ImageQuant software (Molecular Dynamics version 5.2). The results presented in FIG. 3A, 3B, and 3C show a two-fold increase in the level of PKR protein expression in primed and induced PKR overexpressing Namalwa cells (41.027.C1&41.027.A9 ) relative to primed and induced wild type Namalwa cells. Additionally, correlation of the activity data with the relative levels of protein expression reveals an overall three- to five- fold increase in PKR specific activity in primed and induced PKR-overexpressing Namalwa cells compared to primed and induced wild type Namalwa cells (FIG. 4A and FIG. 4B).

### EXAMPLE 3

### Enhanced Interferon Production In A PKR Overexpressing Cell Line In Response To Viral Induction Or Treatment With Poly I:C

Cells from individual subclones of Namalwa PKR++41.027 cells and WT Namalwa cells were pretreated with 20 nM phorbol myristate acetate at a cell density 5.0×10⁵ cells/ml, in 6 well plates for 24 hrs followed by treatment with 500 HAU/ml Sendai virus or 200 μg/mL poly I:C plus 10 μg/mL DEAE Dextran for an additional 72 hr to induce IFN expression. Culture supernatants from the Sendai virus-treated cells were removed at 24 hr and 48 hr post-induction and supernatants from the Poly I:C treated cells were removed at 72 hr. IFN-alpha production was measured in these cell culture supernates using ELISA (Human Interferon Alpha Multi-Species ELISA Kit obtained from PBL).

FIG. 5 illustrates IFN-alpha production was enhanced following both Sendai virus and PolyI:C induction in Namalwa PKR++41.027 cells relative to IFN-alpha production by wild type Namalwa cells as shown in FIG. 5. The level of IFN-alpha production was greatest at 48 hr post Sendai Virus induction, at which point the Namalwa PKR++41.027 cells exhibited approximately four-fold (4×) greater IFN-alpha production than wild type Namalwa cells. Similarly, poly I:C is capable of inducing IFN in the Namalwa PKR ++41.027 cells, despite the absence of detectable IFN-alpha induction in the parental wild type Namalwa cells (FIG. 5).

Parallel experiments were performed on duplicate cultures of the individual subclones of Namalwa PKR++41.027 cells and WT Namalwa cells. These cultures were also pretreated with 20 nM phorbol myristate acetate at a cell density 5.0×10⁵ cells/ml, in 6 well plates for 24 hr followed by treatment with 500 HAU/ml Sendai virus or poly I:C (200 μg/ml) plus 10 μg/ml DEAE Dextran. In addition these cultures were also treated with the PKR inhibitor 5 mM 2-aminopurine (2AP; obtained from Aldrich Chemical Company). Culture supernatants from the Sendai virus-induced cells were removed at 24 hr, post-induction and culture supernatants from the Poly I:C-induced cells were removed at 72 hr and evaluated for IFN alpha production using ELISA (PBL Catalogue No. 41105 Human Interferon Alpha Multi-Species ELISA Kit).

FIG. 5 shows decreased IFN-alpha production following Sendai virus or poly I:C induction in Namalwa PKR++41.027 and WT cells when exposed to 2-aminopurine. The results indicate that 2-aminopurine, a known inhibitor of PKR kinase activity, is capable of inhibiting IFN-alpha induction. Similarly, 2-aminopurine inhibits the induction of other proinflammatory cytokines by virus or poly I:C. (results not shown).

### EXAMPLE 4

### Enhanced TNF-beta Production In A PKR Overexpressing Cell Line In Response To Viral Induction

Cells from individual subclones of Namalwa PKR++41.027 cells and WT Namalwa cells were pretreated with 20 nM phorbol myristate acetate at a cell density 5.0×10⁵ cells/ml, in 6 well plates for 20 hr. This was followed by treatment with 200 μg/ml poly I:C for an additional 72 hrs to induce cytokine expression. Culture supernatants from the treated cells were removed at 72 hrs post induction and evaluated for TNF-beta, IL-6 and IL-8 production via ELISA (R & D Systems).

FIG. 6 illustrates enhanced TNF-beta, IL-6 and IL-8 production following poly I:C induction in Namalwa PKR++41.027 cells relative to TNF-beta, IL-6 and IL-8 production by wild type Namalwa cells. The level of TNF-beta production at 72 hours post-induction in the Namalwa PKR++41.027 cells exhibited approximately 3 fold (3×) greater TNF-beta production than wild type Namalwa cells. Similarly, at 72 ours post-induction with poly I:C, there was more than a 10-fold induction of IL-6 and IL-8 in the 41.027 cells relative to that of the parental controls.

It is claimed:

1. A human cell composition comprising PKR-overexpressing cells, prepared by the process of:
(a) providing a parental cell line capable of expressing PKR and one or more cytokines;
(b) subjecting said parental cell line to limiting dilution cloning in a manner effective to generate a plurality of subclones;
(c) assaying said plurality of subclones for PKR expression; and
(d) selecting a PKR overexpressing subclone identified by a PKR expression level which is at least 2-fold (2×) greater than the PKR expression level of said parental cell line and growing said subclone to generate a PKR overexpressing cell line.

2. The cell composition of claim 1, wherein the preparative process further comprising:
(e) priming said PKR overexpressing cells; and
(f) growing said primed PKR overexpressing cells in a manner effective to generate a PKR overexpressing cell line characterized by enhanced expression of one or more cytokines.

3. The cell composition of claim 2, wherein said preparative process further comprising treating said primed, PKR overexpressing cells before said growing (f).

4. The cell composition of claim 1, wherein said parental cell line is subjected to limiting dilution cloning at least 3 times.

5. The cell composition of claim 2, wherein said priming includes culturing the cells with phorbal myristate acetate (PMA) or sodium butyrate.

6. The cell composition of claim 3, wherein said treating includes non-viral induction with dsRNA.

7. The cell composition of claim 3, wherein said treating includes viral induction with Sendai virus.

8. The cell composition of claim 3, wherein said one or more cytokines are selected from the group consisting of α-interferon, β-interferon, TNF-β, IL-6 and IL-8, IL-10, IFN-gamma, IL-12 and GM-CSF.

9. The cell composition of claim 3, wherein said one or more cytokines include α- or β-interferon.

10. A human cell composition for use in cytokine production, comprising:
PKR overexpressing cells which have been treated and induced in a manner effective to result in enhanced PKR and cytokine production, wherein the cells are characterized by a level of expression or production of one or more cytokines that is at least 2-fold greater than that produced by the corresponding parental cell line.

11. A method for producing a cell line that overexpresses PKR, said method comprising:
(a) providing a parental cell line capable of expressing PKR and one or more cytokines;
(b) subjecting said parental cell line to limiting dilution cloning in a manner effective to generate a plurality of subclones;
(c) assaying said plurality of subclones for PKR expression; and
(d) selecting a PKR overexpressing subclone identified by a PKR expression level which is at least 2-fold (2×) greater than the PKR expression level of said parental cell line; and
(e) growing said subclone to generate a PKR overexpressing cell line.

12. The method according to claim 11 further comprising:
priming said PKR overexpressing cell line before said growing;

13. The method according to claim 12 further comprising:
inducing said primed, PKR overexpressing cell line before said growing.

14. The method of claim 12, wherein said priming includes culturing said PKR overexpressing cells with phorbal myristate acetate (PMA) or sodium butyrate.

15. The method of claim 13, wherein said inducing includes non-viral induction with dsRNA and viral induction with Sendai virus.

16. The method of claim 11, wherein said growing is in a manner effective to generate a PKR overexpressing cell line characterized by enhanced expression of one or more cytokines.

17. The method according to claim 16, wherein said one or more cytokines are selected from the group consisting of α-interferon, β-interferon, TNF-β, IL-6 and IL-8.

18. The method according to claim 16, wherein said one or more cytokines include α- or β-interferon.

19. A human cell composition for enhanced cytokines production comprising PKR-overexpressing cells, prepared by a process comprising:
(a) providing a parental cell line capable of expressing PKR and a heterologous nucleic acid construct that comprises a first coding sequence for a biologically active form of human PKR and a second coding sequence for a selectable marker;
(b) introducing said heterologous nucleic acid sequence construct into said parental cell line in manner effective to result in expression of PKR and said selectable marker;
(c) culturing said transformed parental cells under conditions effective to enhance expression of PKR;
(d) selecting transformed, PKR overexpressing cells, wherein said PKR overexpressing cells are identified by a PKR expression level which is at least 2-fold (2×) greater than the PKR expression level of the non-transformed parental cell line;
(e) growing said selected PKR overexpressing cells; and
(f) repeating said selection and growing to generate PKR overexpressing progeny cell lines characterized by enhanced expression of one or more cytokines.

20. The cell composition of claim 19, wherein said preparative process further comprising:
priming said PKR overexpressing cells and treating said primed, PKR overexpressing cells before said growing.

21. The cell composition of claim 20, wherein said priming includes culturing the cells with phorbal myristte acetate (PMA) or sodium butyrate.

22. The cell composition of claim 20, wherein said treating includes non-viral induction with dsRNA or viral induction with Sendai virus.

23. The cell composition of claim 19, wherein said one or more cytokines are selected from the group consisting of α-interferon, β-interferon, TNF-β, IL-6 and IL-8, IL-10, IFN-gamma, IL-12 and GM-CSF.

24. The cell composition of claim 19, wherein said one or more cytokines include α- or β-interferon.
